(19) United States
(12) Patent Application Publication
(10) Pub. No.: US 2007/0042408 A1
(43) Pub. Date: Feb. 22, 2007

(54) Nucleotide sequence for assessing transmissible spongiform encephalopathies

(76) Inventors: Michael Knoll, Penzberg (DE); Walter Eberle, Bernried (DE); Christoph Seitz, Mittenwald (DE)

Correspondence Address: Roche Diagnostics OPERATIONS Inc., 9115 Hague Road, PO Box 50457, Indianapolis, IN, 46250-0457, US

(21) Appl. No.: 11/495,362

(22) Filed: Jul. 28, 2006

(30) Foreign Application Priority Data
Aug. 2, 2005 (EP) EP 05016740.2
Aug. 26, 2005 (EP) EP 05018546.1

(51) Int. Cl. C12Q 1/68; C12P 19/34

(52) U.S. Cl. 435/6; 435/91.2

(57) ABSTRACT

The present invention is directed to the assessment of transmissible spongiform encephalopathy (TSE) using a sample from a living individual. The assessment is based on the use of an RNA marker molecule in a sample of whole blood. The RNA encodes a hypothetical cystein protease. The invention is further directed to the detection of the marker molecule by means of real-time PCR. The invention provides the use of the nucleotide sequence as a marker in the assessment of TSE, a method for assessing bovine spongiform encephalopathy (BSE) in a bovine animal as well as kits to practice the invention.

### RELATED APPLICATIONS

This application claims priority to European application EP 05016740.2 filed Aug. 2, 2005, and to European application EP 05018546.1 filed Aug. 26, 2005.

### FIELD OF THE INVENTION

The present invention is directed to the assessment of transmissible spongiform encephalopathies (TSE's) using a sample from a living individual. The assessment is based on the use of an RNA marker molecule in a sample of whole blood. The invention is further directed to the detection of the marker molecule by means of real-time polymerase chain reaction (PCR).

### BACKGROUND OF THE INVENTION

Transmissible spongiform encephalopathies (TSE's) consist of a unique group of invariably fatal neurological disorders which affect both human and animals. TSE's are characterized by long presymptomatic incubation periods of months or years. Brain lesions associated with deposits of protease-resistant proteins are a hallmark of clinically manifest TSE disease.

Variant Creutzfeldt-Jakob disease (vCJD) is a rare and fatal human neurodegenerative condition. The classical form, that is to say Creutzfeldt-Jakob disease (CJD), presents as a subacute dementia, evolving over weeks to several months and is accompanied by pyramidal, extrapyramidal, and cerebellar signs. The mean age of death is 57 years but the disease may occur in the late teens and early twenties. In the final stages of the disease, there is an incapacitating dementia, usually with severe myoclonus. Recently, a variant of CJD (vCJD) was described in UK and France with distinct clinical and pathological features which affected younger individuals (average age 29 years, as opposed to 65 years).

As with CJD, vCJD is classified as a TSE because of characteristic spongy degeneration of the brain and its ability to be transmitted. The presumed infectious agent of TSE is often termed PrP^{sc} or PrP^{res} denoting the disease-specific form of the prion protein (PrP). The biological properties of PrP^{sc} in vCJD and BSE share common biological properties e.g. they have similar incubation periods in various kinds of mice and hamsters. TSEs are also known in other animals. For instance, scrapie affects sheep and goats and has been found in many sheep-producing countries throughout the world for over 250 years. Chronic Wasting Disease (CWD) is a contagious fatal TSE in cervids (members of the deer and elk family).

The hypothesis of a link between vCJD and BSE was first raised because of the association of these two TSEs in time and place. More recent evidence supporting a link including identification of pathological features similar to vCJD in brains of macaque monkeys inoculated with BSE. A vCJD-BSE link is further supported by the demonstration that vCJD is associated with a molecular marker that distinguishes it from other forms of CJD and which resembles that seen in BSE transmitted to a number of other species. Studies of the distribution of the infectious agent in the brains of mice artificially infected with tissues from humans with vCJD and cows with BSE showed nearly identical patterns. The most recent and powerful evidence comes from studies showing that the transmission characteristics of BSE and vCJD in laboratory mice are almost identical, strongly indicating that they are due to the same causative agent. In conclusion, the most likely cause of vCJD is exposure to the BSE agent, most plausibly due to dietary contamination by affected bovine central nervous system tissue.

It follows that with regard to the human food chain there is an urgent need for methods to assess a possible TSE infection in the living host. Particularly there is a desire to assess TSE in living animals at a pre-symptomatic stage and before the animals are slaughtered and processed to enter the human food chain. One way to assess TSE is commonly applied when testing slaughtered cattle routinely for bovine spongiform encephalopathy. A brain sample is derived from a sacrificed animal and the presence of PrP^{sc} in the sample is assessed by means of an immunological test such as an ELISA or a Western blot. An alternative approach targets TSE disease specific changes of gene expression. Neurodegeneration in the brain is apparently closely linked with alterations within the members of the cellular defense system of the central nervous system. Notably, increased expression of distinct genes encoding cathepsin enzymes in microglia and/or astrocytes was found to be a correlate of spongiform encephalopathies.

Microglia and astrocytes are both glial cells. Glial cells are the connective tissue cells of the central nervous system (CNS), serving as the supportive structure that holds together and protects neurons. Astrocytes form one particular type of glial cell. They are relatively large with thread-like projections (star shape) that connect with neurons and small blood vessels (capillaries). These projections form part of the blood-brain barrier. This barrier slows or prevents the passage of unwanted substances, such as harmful chemicals, infectious agents, etc., from the bloodstream into the brain. Astrocytes also accumulate in areas where nerves have been damaged (astrocytosis), sealing off these areas. Microglial cells account for approx. 20% of the total glial population in the central nervous system. They are distributed with no significant local differences in the white and gray matters. In contrast to astrocytes they cover non-overlapping territories. They belong to the mononuclear phagocyte system and form the resident macrophages in the brain tissue, the spinal cord and the retina. Their function in the normal neural parenchyma is unknown. However, in various pathologies they form a most reactive sensor to threats to the nervous system and upon stimulation within a few hours they exhibit an activation program. Microglia are highly reactive, mobile and multifunctional immune cells of the CNS that can play a universal role in the defense of the neural parenchyma. Activated microglial cells become immuno-competent and brain macrophages of microglial origin possess cathepsins B and L which render them potentially cytotoxic (Kreutzberg, G. W., Arzneimittelforschung 45 (1995) 357-360).

Intracellular proteinases such as members of the cathepsin families play a role in the process of neuronal cell death. Apparently, this is also the case during neurodegenerative diseases. An increase in cathepsins B, L, and D (lysosomal proteinases) and cathepsin E (non-lysosomal aspartic proteinase) in neurons has been shown in the early stage of neuronal degeneration. An increased level of such proteinases was also found in reactive glial cells and may be involved in the pathogenesis of neurodegenerative disease (Nakanishi, H., and Yamamoto, K., Japanese Journal of Pharmacology 105 (1995) 1-9 [abstract]).

With respect to TSE neurodegeneration, Diedrich, J. F., et al., J. Virol 65 (1991) 4759-4768 report that neuropathological changes in scrapie are associated with a higher level of cathepsin D mRNA. Increased expression was demonstrated by analysis of mRNA isolates from brains of mice experimentally infected with scrapie 16 weeks postinoculation. Furthermore, overexpression of the mRNAs was mapped to astrocytes. Using an experimental scrapie mouse model and cDNA expression arrays together with quantitative RT-PCR, Brown, A. R., et al., Neuropathology and Applied Neurobiology 30 (2004) 555-567 showed increased expression of cathepsin D in hippocampal tissue. Using microarray analysis of gene expression in mouse brains after infection with two different strains of scrapie Xiang, W., et al., J. Virology 78 (2004) 11051-11060 showed significant up-regulation for cathepsins S, H, C, D, and Z. To a much lesser, if not insignificant extent the expression of cathepsins B and L appeared to be higher in infected brains. In an experimental mouse model for CJD expression of the gene encoding cathepsin S was found to be induced significantly in microglia cells of the brain (Baker, C. A., et al., J. Virology 73 (1999) 5089-5097). An increase by the factor of about 2 was measured between 88 and 120 days after inoculation. In a later report by Baker, C. A., et al., J. Virology 76 (2002) 10905-10913 it is disclosed that steady-state levels of mRNA for cathepsin S were increased about 10-fold in CJD-infected microglia which were isolated from mouse brains. In a further study Baker, C. A., and Manuelidis, L., PNAS 100 (2003) 675-679 found enhanced transcription of the genes encoding cathepsins S, L, and H in microglia isolated from CJD-infected mouse brains. However, another study of CJD infected mouse brains by Lu, Z. Y., et al., J. Cellular Biochemistry 93 (2004) 644-652 found that the expression of cathepsin D and, conflicting with the previously cited disclosure, cathepsin L was not altered over the entire course of infection. Dandoy-Dron, F., J. Biol. Chem. 273 (1998) 7691-7697 describe an analysis by means of mRNA differential display of mouse brains infected with scrapie. It was found that, among other transcripts, the cathepsin S mRNA was preferentially expressed giving rise to a higher abundance of the transcript compared to the uninfected controls. When mice were inoculated intracerebrally with the infectious agent that causes BSE, the abundance of cathepsin S mRNA in mouse brain was enhanced likewise.

So far, the state of the art only presents data about increased transcription of cathepsin genes in brain cells, as a result of TSE. Also, the studies of the state of the art regarding marker molecules are confined to artificial disease models in experimental settings. Particularly, in these models the species barrier is crossed. E.g., mice are infected with the scrapie agent from sheep or the vCJD agent from man.

When serving as a marker for the assessment of TSE, cathepsin gene expression is used in the analysis of a brain sample or even a sampled subpopulation of brain cells (microglia). Using whole brain or specific brain cells as sample material, however, necessitates extensive work and safety precautions while preparing the sample material. E.g., the skull of the animal needs to be opened manually and all objects that come in contact with the brain material are at risk of getting contaminated with contagious material.

### SUMMARY OF THE INVENTION

The object of the present invention was to provide a new marker molecule as a means for assessing a TSE infection which can be analyzed in a sample other than brain material. Preferably, the sample can be taken before an individual suspected to suffer from TSE has died or, in the case of an animal, is killed.

Surprisingly the inventors found that the presence of a nucleotide sequence encoding a fragment of a hypothetical cystein proteinase according to SEQ ID NO: 2 in whole blood correlates with TSE infection. Thus, according to the present invention the problem is solved by the use of a nucleotide sequence encoding a fragment of a hypothetical cystein proteinase according to SEQ ID NO: 2 or a nucleotide sequence complementary thereto, as a marker in the assessment of transmissible spongiform encephalopathy (TSE). Further, the invention provides a method for assessing transmissible spongiform encephalopathy (TSE) in a bovine animal by detecting a target RNA, comprising the consecutive steps of (a) providing a sample from the bovine animal, (b) preparing RNA from the sample of step (a), (c) detecting the target RNA in the RNA preparation of step (b), (d) using the result of step (c) to assess TSE in the bovine animal, characterized in that the sample from the bovine animal is whole blood, and the target RNA encodes an amino acid sequence according to SEQ ID NO: 2 or a subfragment thereof comprising 6 or more consecutive amino acids. In addition, the invention provides kits of parts for assessing TSE in a mammal by detecting a target RNA encoding a fragment of the hypothetical cystein proteinase according to SEQ ID NO: 2 or a subfragment thereof.

### BRIEF DESCRIPTION OF THE DRAWINGS

FIG. 1A: Lanes 1 and 21: Size marker, fragment sizes in base pairs are indicated on the left. Lanes 2 to 19 and 22 to 35: Total nucleic acids isolated from whole blood samples obtained from cattle with BSE. Each lane represents a sample taken from an single animal. In the upper part of each lane genomic DNA is visible, the two distinct lower bands correspond to 28S and 18S ribosomal RNAs. Lane 20 and 36: Isolate from HeLa cells (control). The gel was stained using SYBR Green Gel Stain (Molecular Probes).

FIG. 1B: Lanes 1 and 21: Size marker, fragment sizes in base pairs are indicated on the left. Lanes 2 to 13: Total nucleic acids isolated from animals which were BSE suspects but were tested negative post mortem. Lanes 14 to 34: Total nucleic acids isolated from whole blood samples obtained from BSE-negative cattle. Each lane represents a sample taken from an single animal. In the upper part of each lane genomic DNA is visible, the two distinct lower bands correspond to 28S and 18S ribosomal RNAs. The gel was stained using SYBR Green Gel Stain (Molecular Probes).

FIG. 1C: Lanes 1 and 21: Size marker, fragment sizes in base pairs are indicated on the left. Lanes 2 to 19 and lanes 22 to 35: Total RNA isolated from whole blood samples obtained from cattle with BSE. Each lane represents a sample taken from a single animal. Distinct bands correspond to 28S and 18S ribosomal RNAs. Lane 20 and 36: Isolate from HeLa cells (control). The gel was stained using SYBR Green Gel Stain (Molecular Probes).

FIG. 1D: Lanes 1 and 21: Size marker, fragment sizes in base pairs are indicated on the left. Lanes 2 to 13: Total RNA isolated from animals which were BSE suspects but were tested negative post mortem. Lanes 14 to 34: Total RNA isolated from whole blood samples obtained from BSE-negative cattle. Each lane represents a sample taken from an single animal. Distinct bands correspond to 28S and 18S ribosomal RNAs. The gel was stained using SYBR Green Gel Stain (Molecular Probes).

FIG. 2: Agarose gel showing the results of RT-PCR from whole blood samples obtained from a set of 16 BSE-infected or PrP^{sc}-positive cattle and 10 cattle with a negative (PrP^{sc}-negative) result of a post-mortem test on brain stem tissue. The white arrows identify PCR fragments which have been sequenced. A further description of the samples is given in Example 1.
Lane 1: DNA Molecular Weight Marker XIII (50-750 bp), Roche Diagnostics (catalogue no. 1 721 925). Mixture of restriction fragments from a plasmid; fragment sizes are 50, 100, 150, 200, 250, 500, 750 (black arrow), 2642 bp (black arrow).
Lane 2: Control plasmid, there are three bands (amplification products) in the gel due to the high concentration of plasmid DNA in the PCR reaction mixture.
Lane 3: water control (negative control)
Lane 4 to Lane 13: A1 samples
Lane 14: A2-g
Lane 15: A2-h
Lane 16: A2-a
Lane 17: A2-b
Lane 18: A2-c
Lane 19: A2-d
Lane 20: C1-a
Lane 21: DNA Molecular Weight Marker XIII (50-750 bp), Roche Diagnostics (catalogue no. 1 721 925). Mixture of restriction fragments from a plasmid; fragment sizes are 50, 100, 150, 200, 250, 500, 750 (black arrow), 2642 bp (black arrow).
Lane 22: C1-b
Lane 23: C1-c
Lane 24: C1-d
Lane 25: C1-e
Lane 26: C1-f
Lane 27 and Lane 28: B2
Lane 29 and Lane 30: B1
Lane 31: A1 sample spiked with control plasmid (10⁶ dilution)
Lane 32: A1 sample spiked with control plasmid (10⁹ dilution)
Lane 33: C1 sample spiked with control plasmid (10⁶ dilution)
Lane 34: C1 sample spiked with control plasmid (10⁹ dilution)

FIG. 3: Alignment of amino acid sequences of human cathepsin K precursor (AAX09069 (SEQ ID NO: 18), P43235 (SEQ ID NO: 20)), bovine cathepsin K precursor (BT021052, SEQ ID NO: 19), human cathepsin O precursor (P43234 (SEQ ID NO: 22), NP_{—}001325 (SEQ ID NO: 21)), bovine cathepsin K precursor (BT021052), murine cathepsin O precursor (Q8BM88, SEQ ID NO: 23), SEQ ID NO: 2 and the conceptual catalytic center of the hypothetical cystein protease (Cys motif, SEQ ID NO: 24). The asterisk marks the position of the cysteine residue which is a hallmark of the catalytic center in cysteine proteases.

FIG. 4: Nucleotide sequence alignment comparing the nucleotide sequence encoding the Cys motif (SEQ ID NO: 26) with the corresponding sequence encoding the bovine cathepsin K precursor (BT021052 (SEQ ID NO: 27)). The coding strands of the two sequences were aligned with the base triplett encoding the characteristic cysteine residue in the active center of of cysteine proteases. The top line of the sequence alignment is disclosed as SEQ ID NO: 25.

FIG. 5: Results from real-time RT-PCR of total RNA from whole blood samples of cattle using a LIGHTCYCLER instrument and the experimental setting as described in Example 4. Solid lines indicate individual PCR experiments with samples from BSE-infected animals, dotted lines indicate BSE-free animals. The Figure further illustrates the crossing point data given in Table 2. The ordinate indicates the values for fluorescence (F2/Back-F1) as determined by the LIGHTCYCLER instrument, the abscissa indicates cycle numbers. The water control is marked by “H₂O”.

### DETAILED DESCRIPTION OF THE INVENTION

As a consequence of TSE progression the expression of a number of genes is changed in certain cell types. Detection of such changes can be used for diagnosing TSE, thereby offering an alternative to immunological assays for prion protein (PrP^{sc}). The inventors have surprisingly found in cattle that the presence of an RNA sequence according to SEQ ID NO: 1 in whole blood correlates with TSE infection.

Initial screening of total nucleic acids from blood samples revealed a pair of PCR primers according to SEQ ID NO: 3 and SEQ ID NO: 4 which allowed to amplify a target nucleic acid with a size of about 400 bp. The fragment was found only in samples from cattle which were infected with BSE but could not be detected in uninfected animals. Sequencing revealed the nucleotide sequence of SEQ ID NO: 1 comprising 369 bp. The amplified target nucleic acid of the band at approximately 400 bp was verified to be identical in all positive samples where it turned up. A computer-aided search for the largest uninterrupted coding frame revealed an amino acid sequence of 91 residues as given in SEQ ID NO: 2. A homology search indicated that this sequence is a fragment of a novel hypothetical cystein proteinase. On the level of amino acid sequence the fragment according to SEQ ID NO: 2 shows some homology to murine cathepsin O precursor (swissprot accession Q8BM88), human cathepsin O precursor (swissprot accession P43234; Protein database at the National Center for Biotechnology Information (NCBI), accession No. NP_{—}001325), human cathepsin K precursor (swissprot accession P43235), and bovine cathepsin K precursor (encoded by the mRNA according to NCBI Nucleotide accession BT021052; NCBI Protein accession AAX09069). An alignment of the amino acid sequences is shown in FIG. 3. Particularly, the cysteine residue typical for the catalytic center of cysteine proteases appears to be conserved supporting the hypothesis that SEQ ID NO: 2 is a fragment of a cysteine proteinase. Nevertheless, the amino acid sequence of SEQ ID NO: 2 appears to be unique, so far.

Apparently, the mRNA encoding the hypothetical cystein proteinase is a correlate of TSE in cattle. A first embodiment of the invention is therefore the use of a nucleotide sequence encoding a fragment of a hypothetical cystein proteinase according to SEQ ID NO: 2 or a nucleotide sequence complementary thereto, as a marker in the assessment of transmissible spongiform encephalopathy (TSE).

According to the invention it is preferred that a nucleotide sequence encoding a subfragment of SEQ ID NO: 2 comprising 6 or more amino acids is used as a marker for TSE. More preferred is the use of the nucleotide sequence according to SEQ ID NO: 1 or a nucleotide sequence complementary thereto, as a marker in the assessment of transmissible spongiform encephalopathy (TSE). Even more preferred, the nucleotide sequence is used as a marker to assess TSE in a bovine animal. Even more preferred, the nucleotide sequence has a size of about 150-200 bp.

A further embodiment of the invention is a method for assessing TSE in a bovine animal by detecting a target RNA, comprising the consecutive steps of (a) providing a sample from the bovine animal, (b) preparing RNA from the sample of step (a), (c) detecting the target RNA in the RNA preparation of step (b), (d) using the result of step (c) to assess TSE in the mammal, characterized in that the sample from the bovine animal is whole blood, and the target RNA encodes an amino acid sequence according to SEQ ID NO: 2 or a subfragment thereof comprising 6 or more amino acids. Most preferred, the subfragment is the sequence of SEQ ID NO: 17. As shown in FIG. 2, the target RNA of the primer pair according to SEQ ID NOs: 3 and 4 is undetectable in all samples of whole blood from TSE-free, i.e. uninfected individuals. In contrast, the target RNA is detectable in individuals suspected of suffering from TSE (TSE suspects) or individuals already showing clear signs of TSE. It is emphasized that the assessment of TSE according to the invention does not depend on post mortem samples such as, e.g., brain samples. In contrast, the invention provides the means to assess TSE using whole blood samples which can be obtained with great ease from living individuals.

With great advantage the present invention is used for assessing TSE in cattle. Therefore, in a further preferred embodiment of the invention the whole blood sample is taken from a living bovine animal. Even more preferred, the whole blood sample is obtained from an animal which is to proceed to the abattoir and to be processed for the human food chain later on. In this regard, the method of the invention can also be performed in combination with an immunological test detecting infectious prion protein (PrP^{sc}) or protease-resistant prion protein (PrP^{res}).

Before detection of the target RNA the fraction encompassing all nucleic acids (DNA and RNA) is purified from the sample which is a complex mixture of different components. Often, for the first steps, processes are used which allow the enrichment of the nucleic acids. To release the contents of cells, they may be treated with enzymes or with chemicals to dissolve, degrade or denature the cellular walls. This process is commonly referred to as lysis. The resulting solution containing such lysed material is referred to as lysate. A problem often encountered during the lysis is that other enzymes degrading the component of interest, e.g. ribonucleases degrading RNA, come into contact with the component of interest during lysis. These degrading enzymes may also be present outside the cells or may have been spatially separated in different cellular compartiments before the lysis and come now into contact with the component of interest. It is common to use chaotropic agents as e.g. guanidinium thiocyanate or anionic, cationic, zwitterionic or non-ionic detergents when nucleic acids are intended to be set free. It is also an advantage to use proteases which rapidly degrade these enzymes or unwanted proteins. However, this may produce another problem as the said substances or enzymes can interfere with reagents or components in subsequent steps. Proteases (see Walsh, Enzymatic Reaction Mechanisms. W. H. Freeman and Company, San Francisco, Chapter 3 (1979)) which are commonly known to the are e.g. alkaline proteases (WO 98/04730) or acid proteases (U.S. Pat. No. 5,386,024). The protease which is widely used in the prior art for sample preparation for the isolation of nucleic acids is proteinase K from Tritirachium album (see e.g. Sambrook, J. et al., Molecular Cloning: A Laboratory Manual, Cold Spring Harbor Laboratory Press, Cold Spring Harbor, N.Y., 1989) which is active around neutral pH and belongs to a family of proteases known as subtilisins.

In the next steps of the sample preparation which follow on the lysis step, the nucleic acids are further enriched. There are several methods for the extraction of nucleic acids: (A) sequence-dependent or biospecific methods such as e.g.: affinity chromatography and hybridisation to immobilised probes; (B) sequence-independent or physico-chemical methods such as e.g.: liquid-liquid extraction (e.g. with phenol-chloroform), precipitation (e.g. with pure ethanol), extraction with filter paper, extraction with micelle-forming agents (e.g. cetyl-trimethyl-ammonium-bromide), binding to immobilised intercalating dyes (e.g. acridine derivatives), adsorption to silica gel or diatomic earths, and adsorption to magnetic glass particles (MGP) or organo silane particles under chaotropic conditions. Particularly interesting for extraction purposes is the adsorption of nucleic acids to a glass surface although other surfaces are possible. Many procedures for isolating nucleic acids from their natural environment have been proposed in recent years by the use of their binding behavior to glass surfaces. If unmodified nucleic acids are the target, a direct binding of the nucleic acids to a material with a silica surface or glass is preferred because among other reasons the nucleic acids do not have to be modified and even native nucleic acids can be bound. To separate the particles from the contaminants, the particles may be either centrifuged or fluids are drawn through glass fiber filters. This is a limiting step, however, that prevents the procedure from being used to process large quantities of samples. The use of magnetic particles to immobilize nucleic acids after precipitation by adding salt and ethanol is more advantageous and described e.g. in Alderton, R. P., et al., Anal. Biochem. 201 (1992) 166-169 and WO 91/12079. In this procedure, the nucleic acids are agglutinated along with the magnetic particles. The agglutinate is separated from the original solvent by applying a magnetic field and performing washing steps. After these washing steps, the nucleic acids are dissolved in a Tris buffer. Magnetizable particular adsorbents proved to be very efficient and suitable for automatic sample preparation. Ferrimagnetic and ferromagnetic as well as superparamagnetic pigments are used for this purpose. The most preferred magnetic glass particles and methods using these are described in WO 01/37291.

After the purification or isolation of the nucleic acids including the target nucleic acid from their natural surroundings, the target nucleic acid may be detected. Before detection, however, in an additional step the nucleic acids are incubated with RNase-free DNase, thereby allowing to specifically purify the RNAs which were present in the sample. Example 2 describes a preferred method of sample preparation using the MAGNAPURE LC instrument and an RNA isolation kit (Roche Diagnostics GmbH, Mannheim, Germany).

It is particularly advantageous to detect the target RNA by way of specific amplification using RT-PCR (RT=reverse transcriptase; PCR=polymerase chain reaction). The target RNA is first reverse-transcribed to form a complementary single-stranded cDNA. The cDNA in turn serves as a template for DNA amplification using suitable primers. The amplification product is also referred to as the “target nucleic acid”.

Accordingly, a very much preferred embodiment of the invention is a method comprising the steps of (a) providing a whole blood sample from the bovine animal; (b) preparing RNA from the sample of step (a); (c) providing a pair of primers comprising a first and a second primer, whereby the first primer consists of 12 or more contiguous nucleotides of a nucleic acid sequence encoding the amino acid sequence according to SEQ ID NO: 2, and whereby the second primer consists of 12 or more contiguous nucleotides of a nucleic acid sequence complementary to the nucleic acid sequence encoding the amino acid sequence according to SEQ ID NO: 2; (d) amplifying a target nucleic acid from the RNA preparation of step (b) with the primers of step (c); (e) detecting the target nucleic acid of step (d); (f) subsequently using the result of the detection of step (e) to assess TSE in the bovine animal. It is even more preferred that the target nucleic acid is amplified by way of polymerase chain reaction. With great advantage, detection of the target RNA of the invention is therefore achieved using RT-PCR. This method of amplifying a target nucleic acid is also well known to the skilled person. During RT-PCR the target RNA strand is “reverse” transcribed into its DNA complement, followed by amplification of the resulting DNA by means of PCR. Transcribing an RNA strand into its DNA complement is termed reverse transcription (RT), and is accomplished through the use of an RNA-dependent DNA polymerase (reverse transcriptase). Afterwards, a second strand of DNA is synthesized through the use of a deoxyoligonucleotide primer and a DNA-dependent DNA polymerase. The complementary DNA and its anti-sense counterpart are then exponentially amplified. The exponential amplification via RT-PCR provides for a highly sensitive technique, where very low copy number RNAs can be detected.

In a preferred embodiment of the invention the target nucleic acid has the nucleotide sequence of SEQ ID NO: 1 or is a fragment thereof. In the method of the invention a primer pair consisting of a first “forward” primer and a second “reverse” primer is used to amplify SEQ ID NO: 1 or a fragment thereof as well as variants of said nucleotide sequence or fragments thereof. The first and the second primer are oligonucleotides with a preferred length of between 12 and 30 nucleotides, more preferred between 15 and 25 nucleotides. Preferably, the sequence of the first primer is a contiguous partial sequence of the nucleotide sequence of SEQ ID NO: 1. Also preferred, the sequence of the second primer is a contiguous partial sequence of the complement of the nucleotide sequence of SEQ ID NO: 1. Although there are numerous possibilities to design such primers, there are preferred primer sequences and combinations of primers according to the invention. For amplification the first primer is preferably selected from the group consisting of SEQ ID NO: 3, SEQ ID NO: 5, SEQ ID NO: 7, SEQ ID NO: 9, SEQ ID NO: 11, and SEQ ID NO: 13; the second primer is preferably selected from the group consisting of SEQ ID NO: 4, SEQ ID NO: 6, SEQ ID NO: 8, SEQ ID NO: 10, SEQ ID NO: 12, SEQ ID NO: 14, SEQ ID NO: 15, and SEQ ID NO: 16. It is most preferred that the first primer is the nucleotide sequence according to SEQ ID NO: 5, and the second primer is the nucleotide sequence according to nucleotide sequence of SEQ ID NO: 6.

It is noted that the terms “oligonucleotide” and also “polynucleotide” in the context of the present invention summarizes not only (desoxy)-oligo-ribonucleotides, but also all DNA- or RNA-derivatives known in the art like e.g. methyl-phosphonates, phosphothioates, 2′-O-alkyl-derivatives as well as peptide nucleic acids, and analoga comprising modified bases like 7-Deaza-Purines. It is also noted that a primer oligo- or polynucleotide is understood as being capable of serving as a substrate for template-dependent DNA- or RNA-polymerases. By virtue of polymerase activity a primer can be elongated by the addition of nucleoside phosphates or analogues thereof.

In the amplification reaction mixture the preferred initial concentration of each primer is preferably 0.5 μM.

Detection of the amplified target nucleic acid is possible by several means. In a preferred embodiment of the invention, the amplified target nucleic acid is detected using a fluorescent signal. Several detection formats based on target nucleic acid dependent fluorescent signaling have been disclosed, which enable continuous monitoring of the generation of amplification products (reviewed in Wittwer, et al., Biotechniques, 22, (1997) 130-138). These detection formats include but are not limited to (1) to (4):

(1) Use of fluorescent double-stranded DNA recognizing compounds: Since the amount of double stranded amplification product usually exceeds the amount of nucleic acid originally present in the sample to be analyzed, double-stranded DNA specific dyes may be used, which upon excitation with an appropriate wavelength show enhanced fluorescence only if they are bound to double-stranded DNA. Preferably, only those dyes may be used which like SYBR Green I (Molecular Probes), for example, do not affect the efficiency of the PCR reaction. In a very much preferred embodiment of the invention, the target nucleic acid is detected by monitoring the amplification in real time and determining the amount of amplification product after each cycle. The fluorescent signal generated by the incorporated dye can be quantified. Signal strength correlates with the amount of PCR product formed and thus allows quantification of the target nucleic acid after each circle. Therefore, in an even more preferred embodiment of the invention the target nucleic acid is detected by monitoring the amplification in real time and determining the amount of amplification product after each cycle.

(2) Increased fluorescence resonance energy transfer (FRET) upon hybridization: For this detection format, two oligonucleotide hybridization probes each labeled with a fluorescent moiety are used which are capable of hybridizing to adjacent but non overlapping regions of one strand of the amplification product. Preferably, one oligonucleotide is labeled at the 5′ end and the second oligonucleotide is labeled at the 3′ end. When hybridized to the target DNA, the two fluorescent labels are brought into close contact, such that fluorescence resonance energy transfer between the two fluorescent moieties can take place. As a consequence, the hybridization can be monitored through excitation of the donor moiety and subsequent measurement of fluorescence emission of the second acceptor moiety. In a similar embodiment, only one fluorescently labeled probe is used, which together with one appropriately labeled primer may also serve as a specific FRET pair (Bernard, et al., Anal. Biochem. 255 (1998) 101-107). In a very much preferred embodiment of the invention, the target nucleic acid is detected with FRET hybridization probes. Independent from the detection format or fluorescent label, hybridization probes are always polynucleotides having sequences which are completely identical with or exactly complementary to the sequence of the target nucleic acid. Yet, it is also within the scope of the invention if the probes contain one or several mismatches, as long as they are capable of hybridizing to the amplification product under appropriate hybridization conditions. In any case, it has been proven to be particular advantageous, if the sequence identity or complementarity is 100% over a range of at least 10 contiguous residues. Taking onto account the length of the amplified fragments in the method of the invention, the length of the probe does not exceed 40 nucleotides, preferably not more than 30 nucleotides. However, hybridization probes may have 5′ or 3′ overhangs which do not hybridize to the target nucleic acid.

(3) Hydrolysis probes used in TAQMAN instruments: In order to detect the amplification product, a single-stranded hybridization probe is used, which is labeled with a fluorescent entity, the fluorescence emission of which is quenched by a second label on the same probe which may act as a quenching compound. During the annealing step of the PCR reaction, the probe hybridizes to its target sequence, and, subsequently, during the extension of the primer, the DNA polymerase having a 5′-3′-exonuclease activity hydrolyzes the hybridization probe, such that the fluorescent entity is separated from the quencher compound. After appropriate excitation, fluorescence emission can be monitored as an indicator of accumulating amplification product.

(4) Molecular beacons: Similar to hydrolysis probes, a molecular beacon oligonucleotide is labeled with a fluorescent compound and a quencher compound, which due to the secondary structure of the molecule are in dose vicinity to each other. Upon binding to the target DNA, the intramolecular hydrogen bonding is broken, and the fluorescent compound located at one end of the probe is separated from the quencher compound, which is located at the opposite end of the probe (Lizardi et al., U.S. Pat. No. 5,118,801).

Hybridization probes such as hydrolysis probes or molecular beacons may be used for detection. Most preferred, however, are FRET hybridization probes, i.e. a pair of adjacently hybridizing probes, wherein upon hybridization the two fluorescent moieties are brought into close vicinity such that fluorescent resonance energy transfer (FRET) can take place. The term “FRET hybridization probes” therefore is defined as a pair of hybridization probes, each probe carrying a fluorescent compound, which together may act as a FRET pair thus enabling the detection of a nucleic acid, when both probes are hybridized adjacently to a target molecule.

With great advantage, the assay of the invention can be performed on a LIGHTCYCLER instrument (Roche Diagnostics GmbH, Mannheim, Germany) using a pair of FRET hybridization probes labeled with Fluorescein at the 3′ end of the first hybridization probe and with LIGHTCYCLER Red 640 (Roche Diagnostics GmbH, Mannheim, Germany) at the 5′ end of the second hybridization probe.

The skilled person is familiar with the design of hybridization probes for the detection of a target nucleic acid during real time PCR. There is also a wealth of auxiliary means for this purpose such as computer software. An example therefor is Roche LIGHTCYCLER Probe Design Software 2.0 (Roche Diagnostics GmbH, Mannheim; catalogue no. 04 342 054 001) for the design of HYBPROBE and SIMPLEPROBE hybridization probes.

Principally, any kind of quantification method can be applied, however, it has been proven to be advantageous, if methods using an external standard are applied. The external standard itself may be a plasmid or a linearized template with one or more target sequences to be amplified.

In case of quantification of a nucleic acid using external standards, a calibration curve has to be generated. For this calibration curve, known amounts of the target nucleic acid are amplified and the intensity of fluorescent signal is determined as a function of cycle number. After smoothening of the kinetics by a mathematical fit, the first or second maximum of the derivative are calculated. This enables a correlation between the original target concentration and the fractional cycle number of a determined maximum. Subsequently, determination of unknown analyte concentrations may be performed.

In order to eliminate quantification errors originating from different detection sensitivities, it has been proven to be particular advantageous if the same batch of hybridization probe(s) is used for the sample to be analyzed and for the calibration samples.

In addition, kits of parts are contemplated to facilitate practicing the invention. A further embodiment of the invention is therefore a kit of parts for assessing TSE in a mammal by detecting a target RNA, comprising (i) two oligonucleotide primers for amplification of a target nucleic acid, whereby the first primer consists of 12 or more contiguous nucleotides of a nucleic acid sequence encoding the hypothetical protein according to SEQ ID NO: 2, and whereby the second primer consists of 12 or more contiguous nucleotides of a nucleic acid sequence complementary to the nucleic acid sequence encoding the hypothetical protein according to SEQ ID NO: 2, and (ii) an intercalating dye. A SYBR Green dye is very much preferred. Yet, a further embodiment of the invention is a kit of parts for assessing TSE in a mammal by detecting a target RNA, comprising (i) two oligonucleotide primers for amplification of a target nucleic acid, whereby the first primer consists of 12 or more contiguous nucleotides of a nucleic acid sequence encoding the hypothetical protein according to SEQ ID NO: 2, and whereby the second primer consists of 12 or more contiguous nucleotides of a nucleic acid sequence complementary to the nucleic acid sequence encoding the hypothetical protein according to SEQ ID NO: 2, and (ii) a first and a second oligonucleotide hybridization probe labeled with a first and a second fluorescent moiety, respectively, whereby the two oligonucleotide hybridization probes are complementary to adjacent but non overlapping regions of one strand of the target nucleic acid, whereby the first oligonucleotide hybridization probe is labeled at the 5′ end and the second oligonucleotide hybridization probe is labeled at the 3′ end, and whereby the first fluorescent labels are capable of effecting fluorescence resonance energy transfer. Yet, a further embodiment of the invention is a kit of parts for assessing TSE in a mammal by detecting a target RNA, comprising (i) two oligonucleotide primers for amplification of a target nucleic acid, whereby the first primer consists of 12 or more contiguous nucleotides of a nucleic acid sequence encoding the hypothetical protein according to SEQ ID NO: 2, and whereby the second primer consists of 12 or more contiguous nucleotides of a nucleic acid sequence complementary to the nucleic acid sequence encoding the hypothetical protein according to SEQ ID NO: 2, and (ii) a single-stranded hybridization probe which is labeled with a fluorescent entity, the fluorescence emission of which is quenched by a second label on the same probe, whereby the single-stranded hybridization probe is complementary to a region of one strand of the target nucleic acid. Yet, a further embodiment of the invention is a kit of parts for assessing TSE in a mammal by detecting a target RNA, comprising (i) two oligonucleotide primers for amplification of a target nucleic acid, whereby the first primer consists of 12 or more contiguous nucleotides of a nucleic acid sequence encoding the hypothetical protein according to SEQ ID NO: 2, and whereby the second primer consists of 12 or more contiguous nucleotides of a nucleic acid sequence complementary to the nucleic acid sequence encoding the hypothetical protein according to SEQ ID NO: 2, and (ii) a molecular beacon oligonucleotide complementary to a region of one strand of the target nucleic acid, whereby the molecular beacon oligonucleotide is labeled at opposite ends with a fluorescent compound and a quencher compound, which due to the secondary structure of said molecular beacon oligonucleotide are in dose vicinity to each other.

The kits of the invention preferably further comprise a polymerase enzyme with RNA-dependent DNA polymerase (reverse transcriptase) activity, a polymerase enzyme with DNA-dependent DNA polymerase activity, nucleotide triphosphates, buffers, a pair of primers comprising a first and a second primer, whereby the first primer consists of 12 or more contiguous nucleotides of a nucleic acid sequence encoding the amino acid sequence according to SEQ ID NO: 2, and whereby the second primer consists of 12 or more contiguous nucleotides of a nucleic acid sequence complementary to the nucleic acid sequence encoding the amino acid sequence according to SEQ ID NO: 2, a detection probe or sequence-unspecific detection means (intercalating dye, e.g. SYBR Green) and a positive control nucleic acid. It is preferred that the first primer is selected from the group consisting of SEQ ID NO: 3, SEQ ID NO: 5, SEQ ID NO: 7, SEQ ID NO: 9, SEQ ID NO: 11, and SEQ ID NO: 13; the second primer is preferably selected from the group consisting of SEQ ID NO: 4, SEQ ID NO: 6, SEQ ID NO: 8, SEQ ID NO: 10, SEQ ID NO: 12, SEQ ID NO: 14, SEQ ID NO: 15, and SEQ ID NO: 16. It is most preferred that the first primer is the nucleotide sequence according to SEQ ID NO: 5, and the second primer is the nucleotide sequence according to nucleotide sequence of SEQ ID NO: 6.

The following examples, sequence listing and figures are provided to aid the understanding of the present invention, the true scope of which is set forth in the appended claims. It is understood that modifications can be made in the procedures set forth without departing from the spirit of the invention.

### SPECIFIC EMBODIMENTS

### Example 1

### Blood Samples (Cattle)

A first set of samples from cattle, designated “A1”, comprised 10 individuals which suffered from a natural infection with the BSE inducing infective agent (field cases). By way of immunological detection of PrP^{sc} in brainstem (obex) tissue with a officially approved post mortem test a positive test result was established for each A1 animal.

A further set of blood samples, designated “A2”, was obtained from experimentally inoculated animals. These animals were infected by feeding with brain homogenate (100 g or 1 g) obtained from cattle which were naturally infected with BSE. Two samples (“A2-g” and “A2-h”) were from inoculated animals without any sign of BSE. Two samples (“A2-a” and “A2-b”) were from animals which showed definite signs of BSE. Three samples (“A2-c” and “A2-d”) were from animals which showed possible signs of BSE.

Two healthy animals of the negative (i.e. not infected) control group (designated “B2”) were included in the analysis.

A further set of samples (designated “B 1”) was obtained from healthy animals (beef cattle) prior to slaughtering. All B1 animals were tested for BSE by way of immunological detection of PrP^{su} in brainstem using an officially approved post mortem test.

Another set of blood samples derived from cattle which were suspected of being infected with BSE (group designation “C1”). All C1 animals were tested for BSE by way of immunological detection of PrP^{sc} in brainstem using an officially approved post mortem test. With regard to clinical signs of BSE, one animal (“C1-a”) was inconclusive, another (“C1-d”) showed definite signs of BSE. Four other C1 samples came from animals without clinical signs of BSE.

### Example 2

### Nucleic Acid Preparation

RNA extraction from whole blood samples was performed using the MAGNAPURE LC intrument and either (a) the MAGNAPURE LC mRNA isolation kit 1 for blood and blood cells (Roche Diagnostics GmbH, Mannheim, Applied Science catalogue number 03 004 015), (b) the MAGNAPURE LC RNA isolation kit High Performance (Roche Diagnostics GmbH, Mannheim, Applied Science catalogue number 03 542 394), (c) the MAGNAPURE LC total NA isolation kit—large volume (Roche Diagnostics GmbH, Mannheim, Applied Science catalogue number 03 264 793) or (d) MAGNAPURE total NA Isolation kit (Roche Diagnostics GmbH, Mannheim, Applied Science catalogue number 03 038 505). RNA and total NA were isolated according to the instructions of the manufacturer.

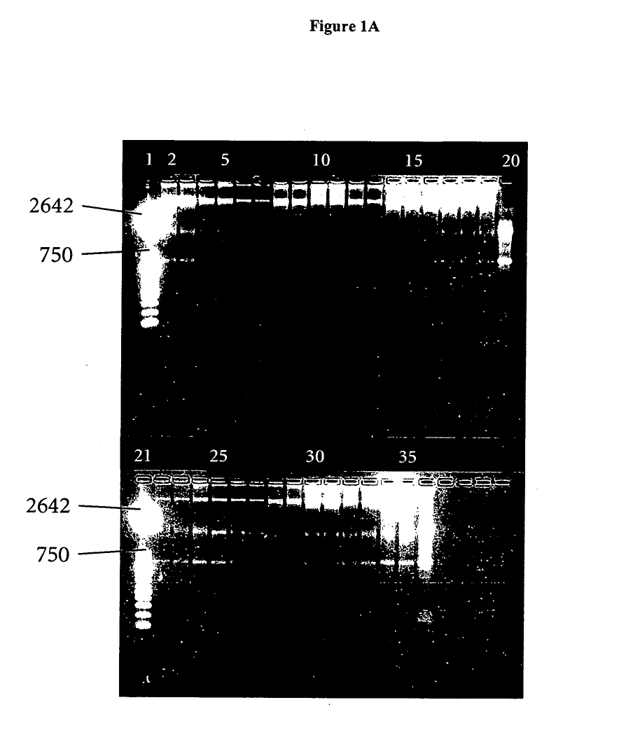

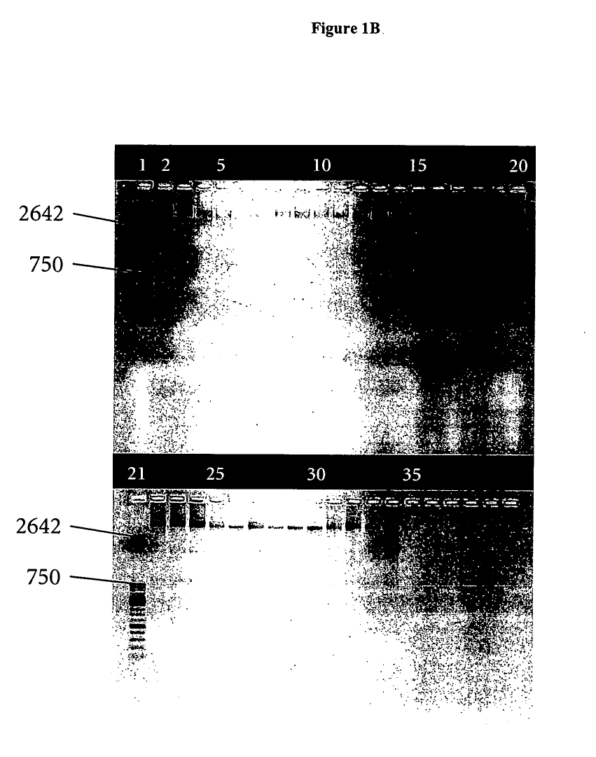

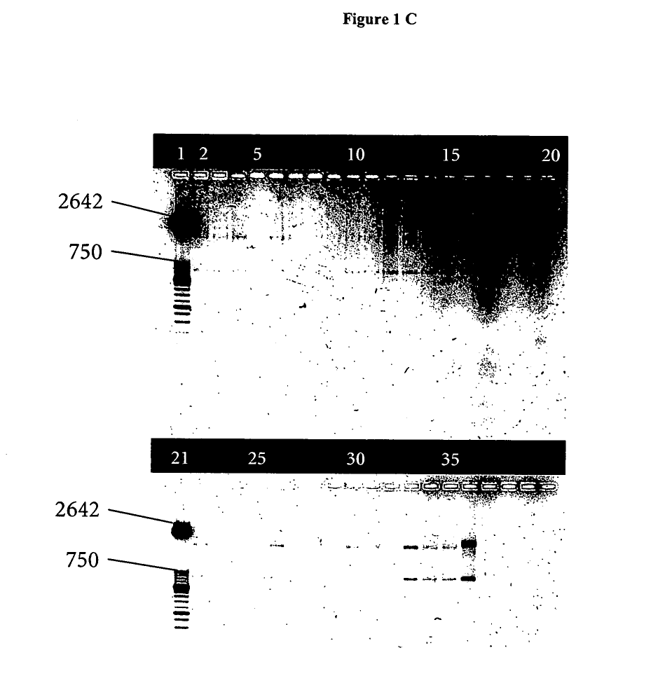

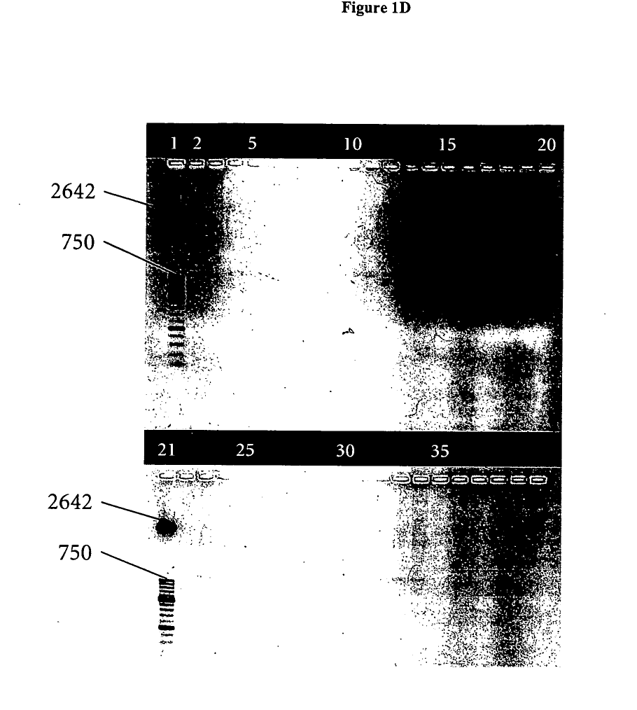

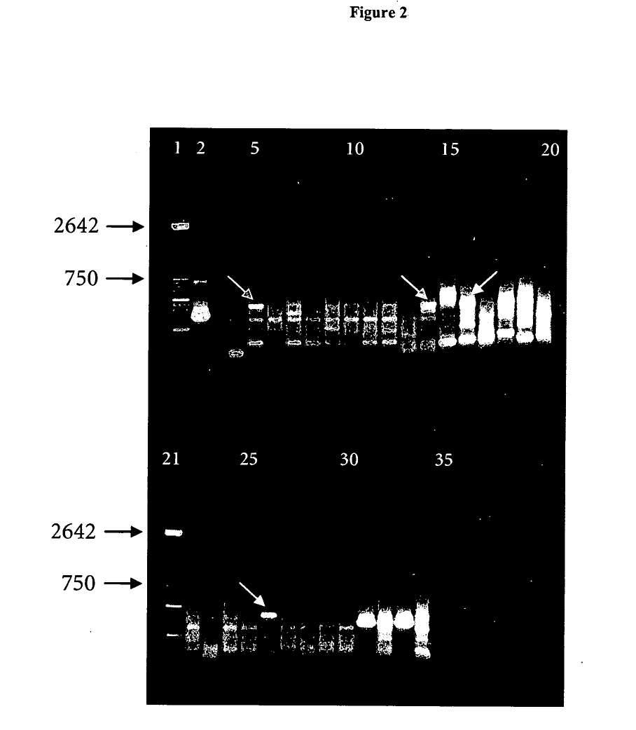

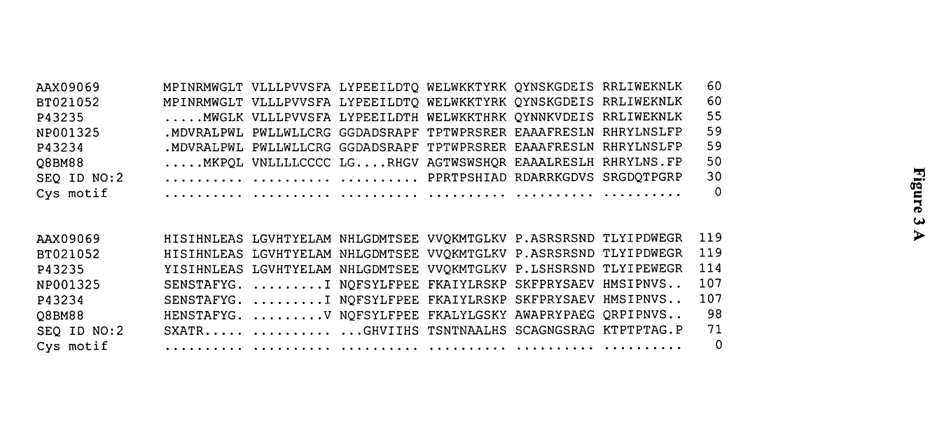

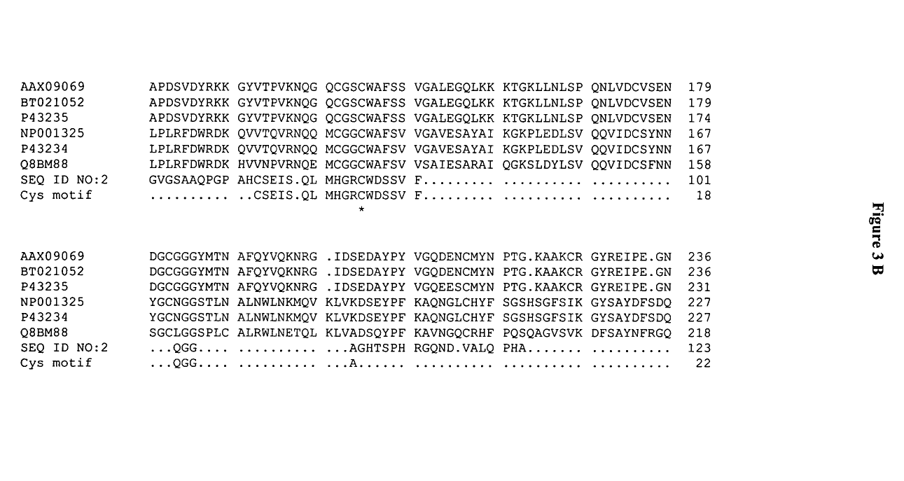

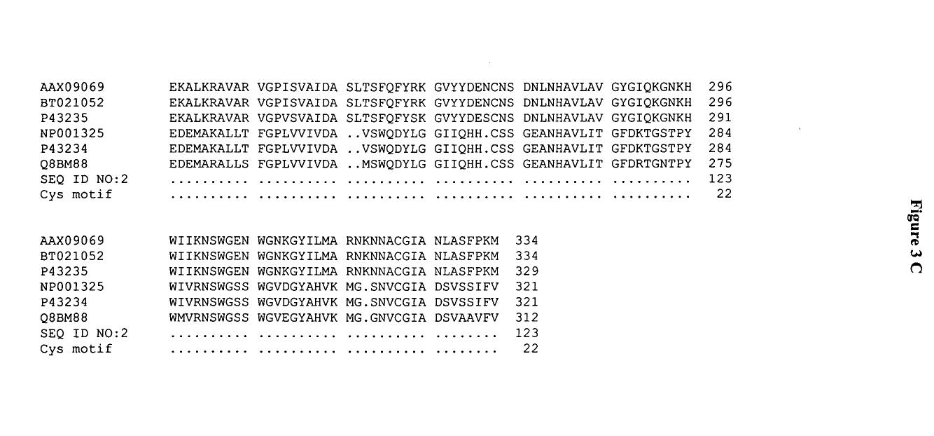

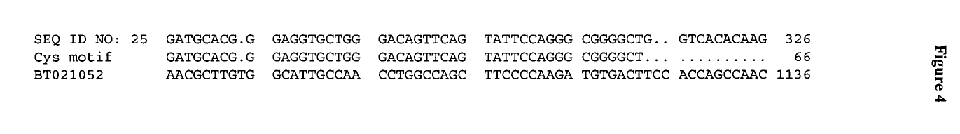

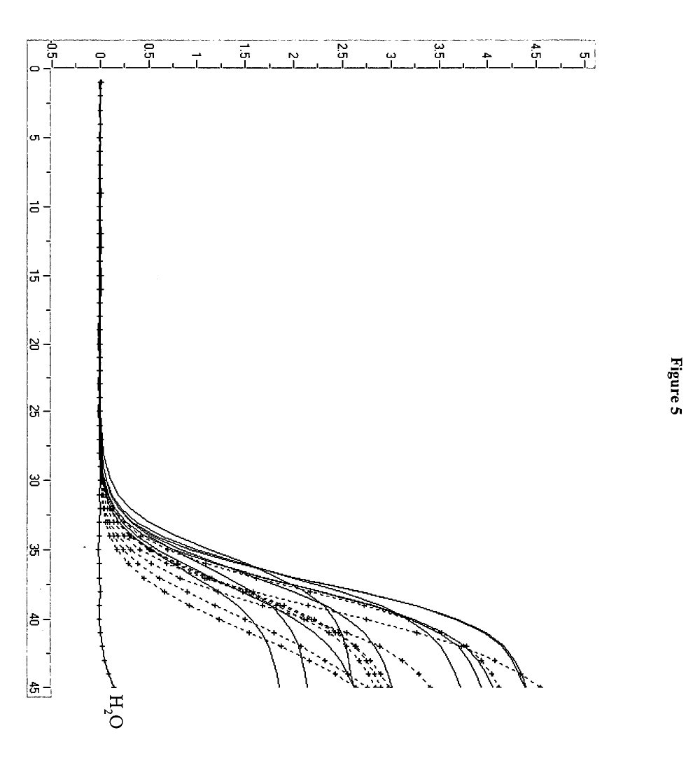

Following purification an aliquot of 5 μl of each preparation (corresponding to 5% of the respective total preparation) was electrophoresed on a 0.8% agarose gel and stained with SYBR Green® I. FIGS. 1 A, B, C, and D shows exemplary agarose electrophoresis gels.

### Example 3

### Pre-Screening for BSE-Specific Marker RNAs and Analysis of Putative Marker Sequences

Light Cycler® RT-PCR was performed using the LC Fast Start DNA Master^{Plus} SYBR Green® I kit (Roche Diagnostics GmbH, Mannheim, Applied Science catalogue no. 03 515 869) and a LIGHTCYCLER 1.2 instrument according to the instructions of the manufacturer.

A first round of screening attempted the detection of BSE-specific RNA sequences in whole blood samples of BSE-infected and not infected cattle. Amplified sequences were characterized and optimized primers were designed and tested. Among a larger collection of primer pairs tested the two oligonucleotides LTR895for and LTR895rev according to SEQ ID NOs: 3 and 4 were characterized in first RT-PCR experiments.

In order to provide positive controls, the target sequences for the two primers were cloned in a plasmid vector (“control plasmid”) in an arrangement such that PCR of the control plasmid yielded an amplified fragment of the plasmid with a size of 350 bp. In control experiments, a solution with a measured amount of isolated control plasmid DNA (Table 1) was used.

TABLE 1
Control plasmid for LTR895 primers, purity
and concentration in aequous solution

| 260 nm | 280 nm | 320 nm | ratio_{260/280} | concentration, in μg/ml |
|---|---|---|---|---|
| 0.062 | 0.034 | 0.001 | 1.823 | 61 |

Extinctions at indicated wavelengths were determined using a NanoDrop ND 1000 UV/Vis spectrophotometer. The conditions for PCR were adjusted using the control plasmid, in order to optimize primer concentration and annealing temperature. In addition, amplification was tested at different concentrations of the control plasmid.

Whole blood RNA preparations from a set of 16 BSE-infected and 10 not infected cattle were then subjected to RT-PCR analysis using the LTR895 primer pair. Resulting amplified DNA fragments were electrophoresed in agarose gels and selected bands of the electrophoretic banding pattern were analyzed. FIG. 2 shows a banding pattern obtained from testing 16 BSE-infected and 10 not infected cattle, together with positive and negative controls.

Four PCR fragments migrating at about 400 bp were isolated and sequenced. Each yielded the nucleotide sequence given in SEQ ID NO: 1. The observation that this fragment turned up in three infected and one clinical BSE suspect case (post-mortem BSE negative) induced further experiments in order to evaluate the diagnostic potential of the target RNA sequence.

### Example 4

### Validation

To prove the potential of the 369 bp RNA fragment according to SEQ ID NO: 1 to discriminate between BSE positive (post-mortem) and healthy cattle primers within SEQ ID NO: 1 were designed for amplification of subfragments. Preferred forward primers were the primers accorsing to SEQ ID NO's: 5, 7, 9, 11, and 13. Preferred reverse primers were the primers accorsing to SEQ ID NO's: 6, 8, 10, 12, 14, 15, and 16. The primer design results in PCR products which are shorter than SEQ ID NO: 1.

PCR was performed as RT-PCR in a Light Cycler® 1.2 instrument using total RNA isolated from whole blood samples and the LC Fast Start DNA Master^{Plus} SYBR Green® I kit (Roche Diagnostics GmbH, Mannheim, Applied Science catalogue no. 03 515 869).

For the respective PCR experiments the primers LTR895(typeI)-1.for and LTR895(typeI)-1.rev according to SEQ ID NO: 5 and SEQ ID NO: 6 were used with particular preference. Real-time PCR analytics with the Roche LIGHTCYCLER 1.2 instrument revealed discrimination of (i) diseased BSE-infected and (ii) healthy animals: BSE animals which were tested as positives post mortem showed a crossing point of 33.2 (mean value) compared to a crossing point of 34.9 (mean value) obtained for healthy individuals (Table 2).

In a PCR amplification reaction, the cycle at which the fluorescence, i.e. in the present case the fluorescence of the complex comprising SYBR Green and the amplified DNA, rises above the background fluorescence is called the “crossing point” of the sample. The crossing point of a sample appears as a sharp upward curve on the experiment's fluorescence chart. The crossing point is the point at which the amplified product is first visible in the data. A sample's crossing point depends on the initial concentration of the target nucleic acid in the sample.

A sample with a lower initial concentration of the target nucleic acid requires more amplification cycles to reach the crossing point. To this end, prior to PCR analysis normalization of nucleic acids was performed to ensure equal RNA amounts in each PCR setup. For nucleic acid normalization the nucleic acid yield of all samples to be compared in a real time PCR run was determined by measuring the OD_{260 nm}. Depending on the resulting nucleic acid yield all samples were diluted to result in identical nucleic acid concentrations. By way of normalization the real-time PCR results of different samples became comparable.

Crossing points determined for individual samples are presented in Table 2. The fluorescence chart of the RT-PCR experiments is given as FIG. 5.

TABLE 2
Crossing points determined in real-time PCR
experiments using the LIGHTCYCLER instrument

| | | Crossing |
|---|---|---|
| Sample | Status | point |
| H₂O | negative run control | >41.00 |
| 1 | infected, clinical signs of BSE, | 32.49 |
| | confirmed by post-mortem test | |
| 2 | infected, clinical signs of BSE, | 32.93 |
| | confirmed by post-mortem test | |
| 3 | infected, clinical signs of BSE, | 33.94 |
| | confirmed by post-mortem test | |
| 4 | infected, clinical signs of BSE, | 33.53 |
| | confirmed by post-mortem test | |
| 5 | infected, clinical signs of BSE, | 33.94 |
| | confirmed by post-mortem test | |
| 6 | infected, clinical signs of BSE, | 33.76 |
| | confirmed by post-mortem test | |
| 7 | infected, clinical signs of BSE, | 31.91 |
| | confirmed by post-mortem test | |
| 8 | infected, clinical signs of BSE, | 33.04 |
| | confirmed by post-mortem test | |
| 9 | infected, clinical signs of BSE, | 33.12 |
| | confirmed by post-mortem test | |
| 10 | infected, clinical signs of BSE, | 33.78 |
| | confirmed by post-mortem test | |
| BSE, average | | 33.2 |
| 11 | healthy, not infected, negative post-mortem test | 35.46 |
| 12 | healthy, not infected, negative post-mortem test | 35.52 |
| 13 | healthy, not infected, negative post-mortem test | 34.07 |
| 14 | healthy, not infected, negative post-mortem test | 34.06 |
| 15 | healthy, not infected, negative post-mortem test | 36.50 |
| 16 | healthy, not infected, negative post-mortem test | 34.04 |
| 17 | healthy, not infected, negative post-mortem test | 35.50 |
| 18 | healthy, not infected, negative post-mortem test | 34.19 |

| | |
|---|---|
| healthy, average | 34.9 |

What is claimed is:

1. A method for assessing transmissible spongiform encephalopathy (TSE) in a bovine animal by detecting a target ribonucleic acid (RNA), comprising the steps of:
(a) providing a sample from the bovine animal,
(b) isolating total RNA from the sample of step (a),
(c) detecting the target RNA in the total RNA isolated in step (b) to obtain a result, and
(d) using the result from step (c) to assess TSE in the bovine animal, wherein the sample from the bovine animal is whole blood, and the target RNA encodes an amino acid sequence according to SEQ ID NO: 2 or a subfragment thereof comprising 6 or more consecutive amino acids.

2. A method for assessing transmissible spongiform encephalopathy (TSE) in a bovine animal by detecting a target ribonucleic acid (RNA), comprising the steps of:
(a) providing a whole blood sample from the bovine animal;
(b) isolating total RNA from the sample of step (a);
(c) providing a first primer consisting of 12 or more contiguous nucleotides of a nucleic acid sequence encoding an amino acid sequence according to SEQ ID NO: 2, and a second primer consisting of 12 or more contiguous nucleotides of a nucleic acid sequence complementary to the nucleic acid sequence encoding the amino acid sequence according to SEQ ID NO: 2;
(d) amplifying a target nucleic acid from the total RNA isolated in step (b) using the first and second primers;
(e) detecting the target RNA amiplified in step (d) to obtain a result; and
(f) using the result from the detection step (e) to assess TSE in the bovine animal.

3. The method of claim 2 wherein the target ribonucleic acid is amplified by way of polymerase chain reaction.

4. The method of claim 2 wherein the amplified target RNA is detected using a fluorescent signal.

5. The method of claim 2 wherein the target RNA is detected by monitoring the amplification in real time and determining the amount of amplification product after each cycle.

6. The method of claim 2 wherein the target RNA consists of the nucleotide sequence of SEQ ID NO: 1 or a subfragment thereof.

7. The method of claim 2 wherein the first primer consists of the nucleotide sequence of SEQ ID NO: 5 and the second primer consists of the nucleotide sequence of SEQ ID NO: 6.

8. A kit of parts for assessing transmissible spongiform encephalopathy (TSE) in a mammal by detecting a target ribonucleic acid (RNA), the kit comprising:
a first oligonucleotide primer for amplification of the target RNA, the first primer consisting of 12 or more contiguous nucleotides of a nucleic acid sequence encoding a hypothetical protein according to SEQ ID NO: 2,
a second oligonucleotide primer for amplification of the target RNA, the second primer consisting of 12 or more contiguous nucleotides of a nucleic acid sequence complementary to the nucleic acid sequence encoding the hypothetical protein according to SEQ ID NO: 2, and
an intercalating dye.

9. A kit of parts for assessing transmissible spongiform encephalopathy (TSE) in a mammal by detecting a target ribonucleic acid (RNA), the kit comprising:
a first oligonucleotide primer for amplification of the target RNA, the first primer consisting of 12 or more contiguous nucleotides of a nucleic acid sequence encoding a hypothetical protein according to SEQ ID NO: 2, and a second primer consisting of 12 or more contiguous nucleotides of a nucleic acid sequence complementary to the nucleic acid sequence encoding the hypothetical protein according to SEQ ID NO: 2, and
a first oligonucleotide hybridization probe labeled with a first fluorescent label and a second hybridization probe labeled with a second fluorescent label, wherein the first and second oligonucleotide hybridization probes are complementary to adjacent but non overlapping regions of the target RNA, wherein the first oligonucleotide hybridization probe is labeled at its 5′ end
and the second oligonucleotide hybridization probe is labeled at its 3′ end, and wherein the first and second fluorescent labels are capable of effecting fluorescence resonance energy transfer.

10. A kit of parts for assessing transmissible spongiform encephalopathy (TSE) in a mammal by detecting a target ribonucleic acid (RNA), the kit comprising:
a first oligonucleotide primer for amplification of the target RNA, the first primer consisting of 12 or more contiguous nucleotides of a nucleic acid sequence encoding a hypothetical protein according to SEQ ID NO: 2, and a second primer consisting of 12 or more contiguous nucleotides of a nucleic acid sequence complementary to the nucleic acid sequence encoding the hypothetical protein according to SEQ ID NO: 2, and
a single-stranded hybridization probe comprising a first and a second fluorescent label, wherein fluorescence emission from the first label is quenched by the second fluorescent label, and wherein the single-stranded hybridization probe is complementary to a region of the target RNA.

11. A kit of parts for assessing transmissible spongiform encephalopathy (TSE) in a mammal by detecting a target ribonucleic acid (RNA), the kit comprising:
a first oligonucleotide primer for amplification of the target RNA, the first primer consisting of 12 or more contiguous nucleotides of a nucleic acid sequence encoding a hypothetical protein according to SEQ ID NO: 2, and a second primer consisting of 12 or more contiguous nucleotides of a nucleic acid sequence complementary to the nucleic acid sequence encoding the hypothetical protein according to SEQ ID NO: 2, and
a molecular beacon oligonucleotide complementary to a region of the target RNA, wherein the molecular beacon oligonucleotide is labeled at one end with a fluorescence emitting compound and at the other end with a fluorescence quenching compound.


SEQUENCE LISTING (27 sequences)

SEQ ID NO: 1 - DNA, 369 bp, Bos taurus
agcgtgtggctggagggcgacgtcattttgtcccctgtgtggacttgtgtgaccagccccgccctggaatactgaactgtcccagcacctcccgtgcatcagttgtgagatctcggagcagtgtgctgggccaggctgggcagctgatccaacccctggccctgcggtgggtgttggggtttttcccgctctggagccgttaccagcacagctgctgtgaagggctgcgttggtattggacgtcgagtgtataatcacatgacctctcgtcgctcaggacggacgcccaggggtctggtcgccccgtgagctcacgtctcctttccttcttgcatctcgatcagcgatgtgtgatggagtcctcggtgg

SEQ ID NO: 2 - protein, 123 aa, Bos taurus
  MOD_RES (32): Variable amino acid
PPRTPSHIADRDARRKGDVSSRGDQTPGRPSXATRGHVIIHSTSNTNAALHSSCAGNGSRAGKTPTPTAGPGVGSAAQPGPAHCSEISQLMHGRCWDSSVFQGGAGHTSPHRGQNDVALQPHA

SEQ ID NO: 3 - DNA, 26 bp, Artificial Sequence
  Description of Artificial Sequence Synthetic primer
tctcaaaacaggcctctgcccggtgg

SEQ ID NO: 4 - DNA, 26 bp, Artificial Sequence
  Description of Artificial Sequence Synthetic primer
tgaggggtcaggagatcccagcgaca

SEQ ID NO: 5 - DNA, 17 bp, Artificial Sequence
  Description of Artificial Sequence Synthetic primer
cctcccgtgcatcagtt

SEQ ID NO: 6 - DNA, 17 bp, Artificial Sequence
  Description of Artificial Sequence Synthetic primer
tgagcgacgagaggtca

SEQ ID NO: 7 - DNA, 17 bp, Artificial Sequence
  Description of Artificial Sequence Synthetic primer
ccgctctggagccgtta

SEQ ID NO: 8 - DNA, 23 bp, Artificial Sequence
  Description of Artificial Sequence Synthetic primer
tgatcgagatgcaagaaggaaag

SEQ ID NO: 9 - DNA, 17 bp, Artificial Sequence
  Description of Artificial Sequence Synthetic primer
tgggcagctgatccaac

SEQ ID NO: 10 - DNA, 23 bp, Artificial Sequence
  Description of Artificial Sequence Synthetic primer
gagatgcaagaaggaaaggagac

SEQ ID NO: 11 - DNA, 17 bp, Artificial Sequence
  Description of Artificial Sequence Synthetic primer
gaagggctgcgttggta

SEQ ID NO: 12 - DNA, 18 bp, Artificial Sequence
  Description of Artificial Sequence Synthetic primer
caccgaggactccatcac

SEQ ID NO: 13 - DNA, 18 bp, Artificial Sequence
  Description of Artificial Sequence Synthetic primer
gagggcgacgtcattttg

SEQ ID NO: 14 - DNA, 17 bp, Artificial Sequence
  Description of Artificial Sequence Synthetic primer
tgagcgacgagaggtca

SEQ ID NO: 15 - DNA, 23 bp, Artificial Sequence
  Description of Artificial Sequence Synthetic primer
ctgatcgagatgcaagaaggaaa

SEQ ID NO: 16 - DNA, 14 bp, Artificial Sequence
  Description of Artificial Sequence Synthetic primer
gctcacggggcgac

SEQ ID NO: 17 - DNA, 189 bp, Bos taurus
cctcccgtgcatcagttgtgagatctcggagcagtgtgctgggccaggctgggcagctgatccaacccctggccctgcggtgggtgttggggtttttcccgctctggagccgttaccagcacagctgctgtgaagggctgcgttggtattggacgtcgagtgtataatcacatgacctctcgtcgctca

SEQ ID NO: 18 - protein, 334 aa, Homo sapiens
MPINRMWGLTVLLLPVVSFALYPEEILDTQWELWKKTYRKQYNSKGDEISRRLIWEKNLKHISIHNLEASLGVHTYELAMNHLGDMTSEEVVQKMTGLKVPASRSRSNDTLYIPDWEGRAPDSVDYRKKGYVTPVKNQGQCGSCWAFSSVGALEGQLKKKTGKLLNLSPQNLVDCVSENDGCGGGYMTNAFQYVQKNRGIDSEDAYPYVGQDENCMYNPTGKAAKCRGYREIPEGNEKALKRAVARVGPISVAIDASLTSFQFYRKGVYYDENCNSDNLNHAVLAVGYGIQKGNKHWIIKNSWGENWGNKGYILMARNKNNACGIANLASFPKM

SEQ ID NO: 19 - protein, 334 aa, Bos taurus; identical to SEQ ID NO: 18

SEQ ID NO: 20 - protein, 329 aa, Homo sapiens
MWGLKVLLLPVVSFALYPEEILDTHWELWKKTHRKQYNNKVDEISRRLIWEKNLKYISIHNLEASLGVHTYELAMNHLGDMTSEEVVQKMTGLKVPLSHSRSNDTLYIPEWEGRAPDSVDYRKKGYVTPVKNQGQCGSCWAFSSVGALEGQLKKKTGKLLNLSPQNLVDCVSENDGCGGGYMTNAFQYVQKNRGIDSEDAYPYVGQEESCMYNPTGKAAKCRGYREIPEGNEKALKRAVARVGPVSVAIDASLTSFQFYSKGVYYDESCNSDNLNHAVLAVGYGIQKGNKHWIIKNSWGENWGNKGYILMARNKNNACGIANLASFPKM

SEQ ID NO: 21 - protein, 321 aa, Homo sapiens
MDVRALPWLPWLLWLLCRGGGDADSRAPFTPTWPRSREREAAAFRESLNRHRYLNSLFPSENSTAFYGINQFSYLFPEEFKAIYLRSKPSKFPRYSAEVHMSIPNVSLPLRFDWRDKQVVTQVRNQQMCGGCWAFSVVGAVESAYAIKGKPLEDLSVQQVIDCSYNNYGCNGGSTLNALNWLNKMQVKLVKDSEYPFKAQNGLCHYFSGSHSGFSIKGYSAYDFSDQEDEMAKALLTFGPLVVIVDAVSWQDYLGGIIQHHCSSGEANHAVLITGFDKTGSTPYWIVRNSWGSSWGVDGYAHVKMGSNVCGIADSVSSIFV

SEQ ID NO: 22 - protein, 321 aa, Homo sapiens; identical to SEQ ID NO: 21

SEQ ID NO: 23 - protein, 312 aa, Mus musculus
MKPQLVNLLLLCCCCLGRHGVAGTWSWSHQREAAALRESLHRHRYLNSFPHENSTAFYGVNQFSYLFPEEFKALYLGSKYAWAPRYPAEGQRPIPNVSLPLRFDWRDKHVVNPVRNQEMCGGCWAFSVVSAIESARAIQGKSLDYLSVQQVIDCSFNNSGCLGGSPLCALRWLNETQLKLVADSQYPFKAVNGQCRHFPQSQAGVSVKDFSAYNFRGQEDEMARALLSFGPLVVIVDAMSWQDYLGGIIQHHCSSGEANHAVLITGFDRTGNTPYWMVRNSWGSSWGVEGYAHVKMGGNVCGIADSVAAVFV

SEQ ID NO: 24 - protein, 22 aa, Artificial Sequence
  Description of Artificial Sequence Synthetic Cys motif
CSEISQLMHGRCWDSSVFQGGA

SEQ ID NO: 25 - DNA, 57 bp, Bos taurus
gatgcacgggaggtgctgggacagttcagtattccagggcggggctggtcacacaag

SEQ ID NO: 26 - DNA, 46 bp, Artificial Sequence
  Description of Artificial Sequence Synthetic oligonucleotide
gatgcacgggaggtgctgggacagttcagtattccagggcggggct

SEQ ID NO: 27 - DNA, 60 bp, Bos taurus
aacgcttgtggcattgccaacctggccagcttccccaagatgtgacttccaccagccaac